(19) United States
(12) Patent Application Publication
(10) Pub. No.: US 2025/0289845 A1
(43) Pub. Date: Sep. 18, 2025

(54) SUBSTRATE FOR SOLID-PHASE ORGANIC SYNTHESIS AND SOLID-PHASE ORGANIC SYNTHESIS METHOD USING THE SAME

(71) Applicant: Sung Koo KANG, Gunpo-si (KR)

(72) Inventor: Sung Koo KANG, Gunpo-si (KR)

(21) Appl. No.: 19/225,378

(22) Filed: Jun. 2, 2025

Related U.S. Application Data
(63) Continuation of application No. PCT/KR2024/002170, filed on Feb. 20, 2024.

(30) Foreign Application Priority Data
Feb. 21, 2023 (KR) 10-2023-0022624
Mar. 7, 2023 (KR) 10-2023-0029768
Mar. 9, 2023 (KR) 10-2023-0031257
Feb. 15, 2024 (KR) 10-2024-0021850

(51) Int. Cl. C07K 1/04; B01J 19/00

(52) U.S. Cl. CPC C07K 1/045; B01J 19/0046; B01J 2219/00495; B01J 2219/00605; B01J 2219/00722; B01J 2219/00725

(57) ABSTRACT

The present invention relates to a substrate for solid-phase organic synthesis and a solid-phase organic synthesis method using the same. More specifically, the present invention relates to a substrate having a larger reactive surface area compared to conventional solid-phase resins, thereby improving the efficiency of solid-phase organic synthesis reactions, and also being applicable to continuous organic synthesis via a roll-to-roll process.

### CROSS-REFERENCE TO RELATED APPLICATIONS

This application is a bypass continuation application of International Application No. PCT/KR2024/002170 field on Feb. 20, 2024, which is based on and claims the benefit of Korean Patent Application No. 10-2023-0022624 filed on Feb. 21, 2023, Korean Patent Application No. 10-2023-0029768 filed on Mar. 7, 2023, Korean Patent Application No. 10-2023-0031257 filed on Mar. 9, 2023, and Korean Patent Application No. 10-2024-0021850 filed on Feb. 15, 2024 in the Korean Intellectual Property Office, the disclosure of each of which is incorporated by reference herein in its entirety.

### BACKGROUND

### 1. Field of the Invention

The present invention relates to a substrate for solid-phase organic synthesis and a solid-phase organic synthesis method using the same. More specifically, the present invention relates to a substrate having a larger reactive surface area compared to conventional solid-phase resins, thereby improving the efficiency of solid-phase organic synthesis reactions, and also being applicable to continuous organic synthesis via a roll-to-roll process.

### 2. Discussion of Related Art

Most organic chemical reactions are carried out in solution. Since typical organic reactions do not proceed with 100% yield, starting materials used at the beginning of the reaction often remain in the reaction mixture after completion. In order to obtain the desired product from the reaction mixture, it is necessary to remove the remaining starting materials and byproducts.

To reduce the likelihood of incomplete reactions, one of the starting materials can be added in excess. For example, if one of two starting materials (A and B) is used in excess (e.g., A), the other starting material (B) may be consumed more completely, minimizing its residue. However, in such cases, the excess starting material (A) and byproducts still need to be removed from the reaction mixture.

Typically, the products of an organic reaction carried out in solution remain dissolved in the solvent. Therefore, to isolate the desired product, separation and purification steps such as crystallization, filtration, recrystallization, extraction, and distillation are required.

Among various types of organic reactions, peptide synthesis is one representative example. Peptide synthesis refers to a reaction that chemically links a plurality of amino acids into a chain of a desired length (e.g., approximately 100 amino acids). Chemical synthesis of peptides has attracted significant interest in various fields, especially due to its ability to produce peptides that do not exist in nature.

Peptide synthesis methods can generally be divided into solution-phase synthesis, where peptide bonds are formed in solution, and solid-phase synthesis, where peptide bonds are added to amino acids immobilized on a solid support such as a resin.

In solution-phase synthesis, as described above, each formation of a peptide bond requires a separation and purification process, which is a major drawback. As a result, most chemical syntheses of peptides nowadays are carried out using solid-phase synthesis, which is relatively simpler and more efficient than solution-phase synthesis.

In solid-phase peptide synthesis, the growing peptide is anchored to a solid support, such as chloromethyl polystyrene resin (Merrifield resin), p-hydroxymethyl phenoxymethyl resin (Wang resin), 2-chlorotrityl chloride resin (2-CTCR), p-methylbenzhydrylamine resin (MBHA resin), or Rink amide AM resin (RAM resin). Generally, the carboxyl terminus (C-terminus) of an amino acid and/or peptide is coupled to the resin, and additional amino acids and/or peptides are successively added to the amino terminus (N-terminus) of the immobilized species to elongate the chain.

Although this solid-phase peptide synthesis technique has been widely used for over 30 years, there remain needs for improvement due to several reasons.

First, conventional solid-phase peptide synthesis is typically performed in a batch reactor, which inherently results in lower yields compared to continuous processes. To form a peptide bond between two amino acid termini, a certain reaction temperature is required. However, when the temperature of the reaction solution in a batch reactor is rapidly increased to accelerate the reaction, it may cause peptide degradation or aggregation of the solid resin.

Furthermore, microwave irradiation is often employed to heat the reaction solution in the batch reactor. As the scale of the batch reactor and the volume of the reaction solution increase to accommodate larger reaction quantities, the temperature of the solution may rise unevenly due to microwave irradiation, leading to reaction non-uniformity. In addition, since the reaction solution must be discarded after the reaction, increasing the volume of solvent leads to greater waste and cost.

Second, the resin serving as the solid support for peptide growth typically has a spherical bead shape. In such cases, peptide growth is limited to the outer surface of the bead, thereby restricting the number of peptides that can be synthesized on a single bead. Even if peptide growth occurs within the interior of the bead, the achievable peptide length is limited.

### SUMMARY OF THE INVENTION

To address the technical problems described above, the present invention provides a substrate for solid-phase organic synthesis that serves as a solid support for such reactions and offers a larger reactive surface area compared to commercially available solid-phase resins.

Another object of the present invention is to provide a substrate for solid-phase organic synthesis that enables continuous organic synthesis using a roll-to-roll process, unlike conventional batch-type solid-phase synthesis methods.

A further object of the present invention is to provide a substrate for solid-phase organic synthesis capable of delivering uniform thermal energy to reactants on the substrate by enabling induction heating of the substrate, instead of heating a reaction solution in a reactor to supply energy for the organic synthesis reaction.

In addition, the present invention provides a method and apparatus for performing organic synthesis using the substrate for solid-phase organic synthesis as defined herein.

It is to be understood that the objects of the present invention are not limited to the purposes mentioned above (e.g., peptide synthesis), and other objects and advantages of the present invention that are not explicitly stated herein will be apparent from the following description. It will also be readily understood that the objects and advantages of the present invention may be realized by means and combinations thereof as defined in the claims.

According to one aspect of the present invention for solving the aforementioned technical problems, there is provided a substrate for solid-phase organic synthesis, comprising: a substrate film; and at least one linker fixed to one side of the substrate film, wherein an organic synthesis reaction is initiated via the linker. The substrate film supplies predetermined thermal energy required for the organic synthesis reaction occurring on the substrate film via the linker by induction heating.

In one embodiment, the substrate may include an induction heating layer that covers at least a portion of one side and the other side of the substrate film and supplies predetermined thermal energy required for the organic synthesis reaction occurring on the substrate film via the linker by induction heating.

In another embodiment, the induction heating layer that supplies predetermined thermal energy required for the organic synthesis reaction occurring on the substrate film via the linker by induction heating may be interposed within the substrate film.

In one embodiment, the induction heating layer may include conductive particles whose temperature can be increased by electromagnetic induction or photothermal conversion.

The conductive particles may include at least one selected from metal powders, metal oxides, metal composite oxides, metal sulfides, metal composite sulfides, ceramic powders, and carbonaceous powders.

The metal may include at least one selected from aluminum, copper, iron, zinc, nickel, gold, silver, tin, platinum, tungsten, cobalt, lead, silicon, germanium, zinc, titanium, and alloys thereof.

In another embodiment, the induction heating layer may be a metal foil comprising at least one selected from: (1) a metal (or a metal in powder form) including at least one selected from aluminum, copper, iron, zinc, nickel, gold, silver, tin, platinum, tungsten, and alloys thereof; and (2) an oxide of at least one selected from aluminum, copper, iron, zinc, nickel, gold, silver, tin, platinum, tungsten, and alloys thereof.

In yet another embodiment, the substrate may include: a first substrate film having at least one linker fixed to one side thereof; a second substrate film having at least one linker fixed to the other side thereof; and an induction heating layer interposed between the first substrate film and the second substrate film to supply predetermined thermal energy required for the organic synthesis reaction occurring via the linker fixed on the first substrate film and the second substrate film by induction heating.

In certain embodiments, the substrate may further include a barrier fixed to one side of the substrate film and provided at a predetermined height adjacent to the linker.

### BRIEF DESCRIPTION OF THE DRAWINGS

FIGS. 1 to 5 schematically illustrate substrates for solid-phase organic synthesis according to some embodiments of the present invention.

### DETAILED DESCRIPTION OF EXEMPLARY EMBODIMENTS

For ease of understanding the present invention, certain terms are defined herein for convenience. Unless otherwise defined, scientific and technical terms used in this specification shall have the meanings generally understood by those of ordinary skill in the art to which the invention pertains.

Unless specifically stated otherwise, the singular forms of terms shall include the plural forms as well, and the plural forms shall include the singular forms.

As used herein, the term “solid-phase organic synthesis reaction” refers to an organic synthesis reaction performed on a solid substrate or support. The term “organic synthesis reaction” refers to a reaction for synthesizing biomolecules such as carbohydrates (e.g., disaccharides, polysaccharides), proteins (e.g., amino acids, peptides), or nucleic acids (e.g., oligonucleotides).

The type of linker in which the organic synthesis reaction occurs, or the type of reactive functional group bound to the linker, may vary depending on the type of organic synthesis reaction. In addition, when the reactive functional group bound to the linker is protected by a protecting group, the type of protecting group may also vary depending on the type of organic synthesis reaction.

When the organic synthesis reaction is a peptide synthesis reaction, the linker may be an amino acid and/or (oligo) peptide suitable for solid-phase peptide synthesis, or another conventionally used linker. Non-limiting examples of such linkers include Rink amide linker, PAL linker, PAM linker, BHA linker, Weinreb linker, and Sieber linker.

When the organic synthesis reaction is a nucleic acid synthesis reaction, the linker may include a functional group selected from: carboxyl, carboxylic acid derivatives, hydroxyl, haloalkyl, dienophile, aldehyde, substituted ketone, sulfonyl halide, thiol, unsubstituted amino, primary amino, alkene, alkyne, halogen, hydrazide, azide, imide, ketene, isocyanate, thiocyanate, isothiocyanate, epoxide, maleimide, 1,2,4,5-tetrazine derivatives, cycloalkyne derivatives, cycloalkene, triphosphate, phosphoramidite, substituted thioketone, halofomyl, formyl, acyl, carboxyl, acylamide, acylazide, organic acid anhydride, aniline, aziridine, boronate, carbodiimide, diazoalkyne, haloacetamide, imido ester, glycol, halotriazine, hydrazine, acyl halide, alkyl halide, and aryl halide, or any other functional group substituted with one or more of the foregoing.

Referring to FIG. 1, FIG. 1 schematically illustrates a substrate for solid-phase organic synthesis (100; hereinafter referred to as “substrate 100”) according to one embodiment of the present invention.

As shown in FIG. 1, the substrate 100 includes a substrate film 110 and at least one linker 130, which is fixed to one side of the substrate film 110 and at which an organic synthesis reaction is initiated.

The substrate film 110 serves as a material for maintaining the basic shape of the substrate 100 and providing a region where the linker 130, which initiates the organic synthesis reaction, is bound. The type of material for the substrate film 110 is not particularly limited.

Examples of materials that may be used to form the substrate film 110 include polymer resins, such as polyester, polyethylene terephthalate (PET), polycarbonate, polymethyl methacrylate (PMMA), polyethylene naphthalate (PEN), polysulfone, polyethersulfone, cyclic olefin polymers, triacetyl cellulose (TAC), polyvinyl alcohol (PVA), polyetherketone, polyarylate, polyimide, and polystyrene, or glass.

When the substrate 100 is used in a continuous roll-to-roll device, it may be preferable for the substrate film 110 to be made of a polymer resin in film form rather than glass.

The thickness of the substrate film 110 is not particularly limited, but it is preferably in the range of 10 μm to 200 μm in order to improve handling convenience.

To supply a predetermined amount of thermal energy required for the organic synthesis reaction by induction heating, an induction heating layer 120 may be attached to at least a portion of the other side of the substrate film 110. The linker 130 may be fixed to the substrate film 110 and/or the induction heating layer 120.

Unless otherwise defined, the term “induction heating” as used herein includes both temperature increases caused by electromagnetic induction and photothermal conversion.

For example, the induction heating layer 120 may be provided to cover one side of the substrate film 110 with respect to a region in which the linker 130 is fixed on the substrate film 110. If the linker 130 is disposed over the entire surface of the substrate film 110, the induction heating layer 120 may be provided to entirely cover one side of the substrate film 110.

The thickness of the induction heating layer 120 is not particularly limited, but it is preferably in the range of 5 μm to 100 μm so that sufficient thermal energy required for the organic synthesis reaction can be supplied by induction heating. It is also preferable that the induction heating layer 120 be thinner than the substrate film 110.

If the thickness of the induction heating layer 120 becomes excessively large relative to the thickness of the substrate film 110 (for example, if the ratio of the thickness of the induction heating layer 120 to that of the substrate film 110 exceeds 0.5), there is a risk that excessive thermal energy may be supplied by the induction heating layer 120, which may cause damage to the substrate 100 for solid-phase organic synthesis or result in failure to induce the intended organic synthesis reaction.

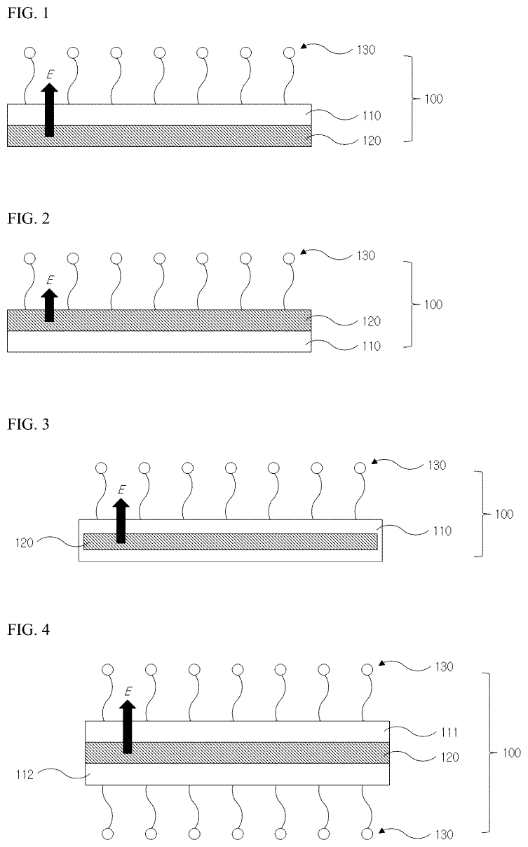

Meanwhile, as shown in FIG. 2, when the side of the substrate film 110 to which the linker 130 is fixed is the same side on which the induction heating layer 120 is located, the linker 130 may be fixed to the induction heating layer 120, or it may be fixed to the substrate film 110 in a state penetrating through the induction heating layer 120.

In one embodiment, the induction heating layer may be a polymer resin layer in which particles capable of increasing in temperature via electromagnetic induction or photothermal conversion (hereinafter referred to as “conductive particles”) are dispersed. That is, the conductive particles may be electrically conductive particles that can be heated by electromagnetic induction, or thermally conductive particles that can be heated by photothermal conversion.

The conductive particles may include at least one selected from metal powders, metal oxides, metal composite oxides, metal sulfides, metal composite sulfides, ceramic powders, and carbon-based powders.

The metal may include at least one selected from aluminum, copper, iron, zinc, nickel, gold, silver, tin, platinum, tungsten, cobalt, lead, silicon, germanium, titanium, and alloys thereof.

In addition, the metal oxide or metal composite oxide may include at least one oxide selected from oxides of aluminum, copper, iron, zinc, nickel, gold, silver, tin, platinum, tungsten, cobalt, lead, silicon, germanium, titanium, and alloys thereof.

The metal sulfide or metal composite sulfide may include at least one sulfide selected from sulfides of copper, iron, zinc, nickel, silver, tin, cobalt, lead, indium, gallium, molybdenum, and alloys thereof.

The carbon-based powder may include at least one selected from carbon black, carbon nanotubes, and carbon nanofibers.

The average particle diameter of the conductive particles may be in the range of 10 nm to 100 μm. If the average particle diameter is smaller than 10 nm, the effect of temperature increase by electromagnetic induction or photothermal conversion may be insignificant. On the other hand, if the average particle diameter exceeds 100 μm, it may be difficult to achieve uniform temperature increase by electromagnetic induction or photothermal conversion due to poor dispersibility of the conductive particles within the induction heating layer 120.

In addition, the induction heating layer 120 may include a first conductive particle and a second conductive particle having different average particle diameters. By using the first conductive particle and the second conductive particle with different average particle sizes, the second conductive particles, having a relatively smaller average diameter, may fill the gaps between the first conductive particles, which have a relatively larger average diameter. As a result, the dispersibility of the conductive particles within the induction heating layer 120 may be improved.

In addition, when the second conductive particles are present in the gaps between the first conductive particles having a relatively larger average particle diameter, temperature rise efficiency may be enhanced through induction heating caused by interaction between the first conductive particle and the second conductive particle during electromagnetic induction or photothermal conversion.

The average particle diameter of the first conductive particles may be from 100 nm to 100 μm, preferably from 100 nm to 0.5 μm, and the average particle diameter of the second conductive particles may be less than 100 nm, preferably 50 nm or less.

To optimize the temperature rise effect achieved by mixing the first conductive particle and the second conductive particle, it is preferable that the induction heating layer 120 includes the first and second conductive particles in a weight ratio of 20:80 to 90:10.

The second conductive particles may be located in the gaps between the first conductive particles, attached to the surfaces of the first conductive particles, or present in an aggregated form with one another.

If the proportion of the second conductive particles relative to the first conductive particles in the induction heating layer 120 is excessively low, or if the proportion of the first conductive particles relative to the second conductive particles is excessively low, the temperature rise effect may be insignificant.

In addition, the induction heating layer 120 may include 5 wt % to 15 wt % of conductive particles. If the content of the conductive particles is less than 5 wt %, the temperature rise effect by electromagnetic induction or photothermal conversion may be insufficient. On the other hand, if the content exceeds 15 wt %, uniform temperature increase by electromagnetic induction or photothermal conversion may be difficult to achieve due to deterioration in the dispersibility of the conductive particles within the induction heating layer 120.

In one embodiment, when the conductive particles dispersed in the induction heating layer 120 are particles capable of increasing in temperature via photothermal conversion, the conductive particles may be responsive to photothermal conversion in a wavelength range of 300 nm to 1,300 nm, preferably 600 nm to 1,300 nm, and more preferably 780 nm to 1,000 nm.

In other embodiment, when the conductive particles are particles capable of increasing in temperature via electromagnetic induction—more specifically, via an alternating magnetic field generated in high-frequency induction heating—the conductive particles may be responsive to induction heating at a frequency in the range of 1 kHz to 40 MHz, preferably 1 kHz to 10 MHz, more preferably 100 kHz to 1 MHz, and most preferably 20 kHz to 100 kHz.

The polymer resin used to form the induction heating layer 120 may include, for example, polyester, polyethylene terephthalate (PET), polycarbonate, polymethyl methacrylate (PMMA), polyethylene naphthalate (PEN), polysulfone, polyethersulfone, cyclic olefin polymers, triacetyl cellulose (TAC), polyvinyl alcohol (PVA), polyether ketone (PEK), polyarylate, polyimide, and/or polystyrene.

In another embodiment, the induction heating layer 120 may be composed of the conductive particles. That is, the induction heating layer 120 may not be formed by dispersing the conductive particles in a polymer resin and coating the mixture onto the surface of the substrate film 110, but rather may be formed by applying a paste containing the conductive particles directly onto the surface of the substrate film 110.

For example, the induction heating layer 120 may be in the form of a metal foil that includes (1) a metal (or a powder thereof) comprising at least one selected from aluminum, copper, iron, zinc, nickel, gold, silver, tin, platinum, tungsten, and alloys thereof; and/or (2) at least one oxide of aluminum, copper, iron, zinc, nickel, gold, silver, tin, platinum, tungsten, or alloys thereof. Although not explicitly illustrated, the metal foil may be provided in a laminated form on one side of a polymer layer other than the substrate film 110.

According to the embodiment illustrated in FIG. 3, the induction heating layer 120 may be interposed within the substrate film 110. In such a configuration, the induction heating layer 120, being embedded within the substrate film 110, may supply a predetermined amount of thermal energy required for the organic synthesis reaction occurring on the substrate film 110.

In a similar example, electrically conductive particles capable of increasing in temperature via electromagnetic induction and/or thermally conductive particles capable of increasing in temperature via photothermal conversion may be dispersed within the substrate film 110. In this case, it is possible to supply a predetermined amount of thermal energy required for the organic synthesis reaction occurring on the substrate film 110 through induction heating, without laminating a separate induction heating layer 120 on one or both sides of the substrate film 110.

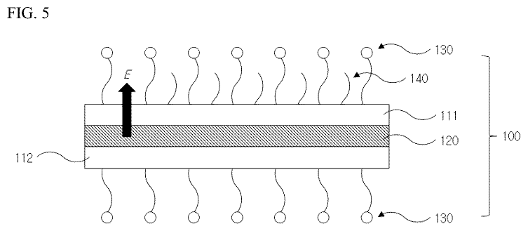

According to the embodiment illustrated in FIG. 4, a first substrate film 111 and a second substrate film 112, each having the linker 130 fixed thereto, may be laminated on opposite sides of a single induction heating layer 120. In this configuration, the efficiency of the organic synthesis reaction can be improved by allowing the reaction to be performed on both sides via the single induction heating layer 120.

According to the embodiment illustrated in FIG. 5, the substrate 100 may further include a non-reactive barrier 140 provided on one side of the substrate films 111 and 112, positioned adjacent to the linker 130 and having a predetermined height. In this case, it is preferable that the length of the barrier 140 be shorter than the length of the linker 130.

The barrier 140 serves to prevent aggregation of reaction products resulting from organic synthesis reactions mediated between adjacent linkers. Accordingly, the barrier 140 is preferably formed of a material that is inert to the organic synthesis reaction. For example, the barrier 140 may be selected from C₁-C₄₀ alkyl, C₁-C₄₀ heteroalkyl, C₂-C₄₀ alkenyl, C₂-C₄₀ alkynyl, C₃-C₂₀ cycloalkyl, C₃-C₂₀ cycloalkenyl, C₂-C₂₀ heterocycloalkyl, C₅-C₅₀ aryl, C₂-C₅₀ heteroaryl, and C₅-C₅₀ aralkyl groups.

As described above, the substrate 100 according to various embodiments of the present invention can locally supply a predetermined amount of thermal energy required for the organic synthesis reaction-initiated via the linker 130 on the substrate film 110—to the surface of the substrate film 110 and adjacent regions, through direct or indirect induction heating of the substrate film 110.

Accordingly, sufficient organic synthesis can be induced on the substrate film 110 while minimizing the need to heat the reaction solution within the reactor in which the organic synthesis reaction is carried out.

The following specific embodiments of the present invention are provided by way of example and illustration only. It should be understood that these embodiments are not intended to limit the scope of the invention in any way.

### Preparation Example 1. Preparation of Substrate a for Solid-Phase Organic Synthesis

### Example 1

### Step 1. Synthesis of Conductive Particles

(a) Silver nitrate (AgNO₃) solution (25 mM, 10 mL) and tri-sodium citrate dihydrate solution (1 wt %, 10 mL) were dissolved in 80 mL of water, and the mixture was reacted for 15 minutes, followed by cooling to synthesize silver nanoparticles having an average particle diameter of 60 nm.

(b) The silver nanoparticle solution (20 mL) prepared in step (a) was diluted with 80 mL of water, followed by the addition of chloroauric acid (HAuCl₄) solution (20 mM, 16 mL) and ascorbic acid solution (10 mM, 16 mL). The mixture was stirred at 600 rpm for 20 minutes. Then, 2 mL of polyvinylpyrrolidone (10 wt %), serving as a surface capping agent, was added, and the reaction was carried out at 58° C. for 24 hours to synthesize hollow-structured gold nanoparticles.

### Step 2. Preparation of Coating Composition

A coating composition was prepared by dissolving the following components in toluene (purity ≥99%, 10 g): 3 g of acrylamide monomer as a monomer, 10 g of 1,3-butanediol dimethacrylate and 10 g of 1-(acryloyloxy)-3-(methacryloyloxy)-2-propanol as crosslinkers, and 0.05 g of 2-hydroxy-2-methylpropiophenone as a photoinitiator. The mixture was stirred at 1000 rpm for 10 minutes to dissolve the components completely.

Subsequently, 0.1 g of the hollow-structured gold nanoparticles synthesized in Step 1 was added to the solution, followed by stirring at 600 rpm for 10 minutes to prepare the coating composition.

### Step 3. Formation of Induction Heating Layer on Substrate Film

The coating composition prepared in Step 2 was applied onto a 50 μm thick polyethylene (PE) film (substrate film) by bar coating, followed by UV curing at 1,000 mJ/cm² to form an induction heating layer having a thickness of 20±5 μm.

### Step 4. Immobilization of Linker for Organic Synthesis Reaction

On the surface of the induction heating layer prepared in Step 3, amino groups derived from the acrylamide monomer were exposed.

An Fmoc-Rink linker, which is used as a linker for organic synthesis reactions, contains an amino group protected by an Fmoc group and a terminal carboxyl group. The carboxyl group of the Fmoc-Rink linker can form an amide bond with the exposed amino group on the surface of the induction heating layer, resulting in the chemical immobilization of the Fmoc-Rink linker onto the surface.

The Fmoc-Rink linker was dissolved in N,N-dimethylformamide (DMF) at a concentration of 3 wt %, and the substrate prepared in Step 3 was immersed in the solution and reacted at 50° C. for 20 minutes to obtain a substrate having the Fmoc-Rink linker chemically bonded to the surface of the induction heating layer.

### Example 2

A substrate for solid-phase organic synthesis was prepared in the same manner as in Example 1, except that 0.2 g of hollow-structured gold nanoparticles was used in Step 2.

### Comparative Example 1

### Step 1. Preparation of Coating Composition

A coating composition was prepared by dissolving 30 g of acrylamide monomer as a monomer, 100 g of 1,3-butanediol dimethacrylate and 100 g of 1-(acryloyloxy)-3-(methacryloyloxy)-2-propanol as crosslinkers, and 1 g of 2-hydroxy-2-methylpropiophenone as a photoinitiator in 100 g of toluene (purity ≥99%). The mixture was stirred at 1000 rpm for 10 minutes to dissolve the components completely.

### Step 2. Formation of Coating Layer

The coating composition prepared in Step 1 was applied onto a 50 μm thick polyethylene (PE) film (substrate film) by bar coating, followed by UV curing at 1,000 mJ/cm² to form a coating layer having a thickness of 15 μm.

### Step 3. Immobilization of Linker for Organic Synthesis Reaction

The linker for the organic synthesis reaction was immobilized on the coating layer in the same manner as in Step 4 of Example 1.

### Reference Example 1

### Step 1. Synthesis of Conductive Particles

Silver nanoparticles having an average particle diameter of 60 nm were synthesized by dissolving silver nitrate (AgNO₃) solution (25 mM, 10 mL) and tri-sodium citrate dihydrate solution (1 wt %, 10 mL) in 80 mL of water, followed by reacting the mixture for 15 minutes and then cooling.

### Step 2. Preparation of Coating Composition

A coating composition was prepared by dissolving 30 g of acrylamide monomer as a monomer, 100 g of 1,3-butanediol dimethacrylate and 100 g of 1-(acryloyloxy)-3-(methacryloyloxy)-2-propanol as crosslinkers, and 1 g of 2-hydroxy-2-methylpropiophenone as a photoinitiator in 100 g of toluene (purity ≥99%) with stirring at 1000 rpm for 10 minutes. Subsequently, 0.1 g of silver nanoparticles synthesized in Step 1 was added to the solution, and the mixture was stirred at 1000 rpm for an additional 10 minutes to obtain the coating composition.

### Step 3. Formation of Induction Heating Layer on Substrate Film

The coating composition prepared in Step 2 was cast onto a 50 μm thick polyethylene (PE) film (substrate film), followed by UV curing at 1,000 mJ/cm² to form an induction heating layer having a thickness of 15 μm.

### Step 4. Immobilization of Linker for Organic Synthesis Reaction

The linker for the organic synthesis reaction was immobilized on the coating layer in the same manner as in Step 4 of Example 1.

### Preparation Example 2. Preparation of Substrate B for Solid-Phase Organic Synthesis

### Preparation Example 2-1

(a) 1 mL of CuCl₂ solution (0.5 mol/L) and 3.6 g of poly(vinylpyrrolidone) were added to 250 mL of distilled water, and the mixture was stirred at room temperature for 10 minutes. Subsequently, 1 mL of Na₂S solution (0.5 mol/L) was added, and the resulting mixture was stirred at room temperature for an additional 10 minutes to prepare a reaction solution.

(b) The reaction solution prepared in step (a) was reacted at 75° C. for 1 hour, followed by centrifugation at 10,000 rpm for 10 minutes. The resulting precipitate was washed with distilled water and then centrifuged again at 10,000 rpm for 10 minutes. The separated CuS nanoparticles were dried at 40° C. The average particle diameter of the CuS nanoparticles obtained in step (b) was confirmed to be approximately 3 nm.

### Preparation Example 2-2

(a) 3 mL of CuCl₂ solution (0.5 mol/L) and 3.6 g of poly(vinylpyrrolidone) were added to 250 mL of distilled water, and the mixture was stirred at room temperature for 10 minutes. Subsequently, 3 mL of Na₂S solution (0.5 mol/L) was added, and the resulting mixture was stirred at room temperature for an additional 10 minutes to prepare a reaction solution.

(b) The reaction solution prepared in step (a) was reacted at 75° C. for 1 hour, followed by centrifugation at 10,000 rpm for 10 minutes. The resulting precipitate was washed with distilled water and then centrifuged again at 10,000 rpm for 10 minutes. The separated CuS nanoparticles were dried at 40° C. The average particle diameter of the CuS nanoparticles obtained in step (b) was confirmed to be approximately 150 nm.

### Example 3

### Step 1. Preparation of Coating Composition

3 g of acrylamide monomer, 10 g of 1,3-butanediol dimethacrylate and 10 g of 1-(acryloyloxy)-3-(methacryloyloxy)-2-propanol as crosslinkers, and 0.05 g of 2-hydroxy-2-methylpropiophenone as a photoinitiator were added to 10 g of toluene (purity ≥99%) and stirred at 1000 rpm for 10 minutes to dissolve the components.

Subsequently, 0.1 g of CuS (average particle diameter: 3 nm) synthesized in Preparation Example 2-1 and 0.1 g of CuS (average particle diameter: 150 nm) synthesized in Preparation Example 2-2 were added to the solution, followed by stirring at 600 rpm for 10 minutes to prepare a coating composition.

### Step 2. Formation of Induction Heating Layer on Substrate Film

The coating composition prepared in Step 1 was applied onto a 50 μm thick polyethylene (PE) film (substrate film) by bar coating, followed by UV curing at 1,000 mJ/cm² to form an induction heating layer having a thickness of 20±5 μm.

### Step 3. Immobilization of Linker for Organic Synthesis Reaction

On the surface of the induction heating layer prepared in Step 2, amino groups derived from the acrylamide monomer are present in an exposed state.

The Fmoc-Rink linker, used as a linker for the organic synthesis reaction, includes an Fmoc-protected amine group and a terminal carboxyl group. The carboxyl group of the Fmoc-Rink linker can form an amide bond with the amino group exposed on the surface of the induction heating layer, thereby allowing the Fmoc-Rink linker to be chemically bonded to the surface of the induction heating layer.

To achieve this, 3 wt % of Fmoc-Rink linker was dissolved in N,N-dimethylformamide, and the substrate prepared in Step 2 was immersed in the solution and reacted at 50° C. for 20 minutes to prepare a substrate in which the Fmoc-Rink linker was chemically bonded to the surface of the induction heating layer.

### Example 4

A substrate for solid-phase organic synthesis was prepared in the same manner as in Example 3, except that 3 g of 2-aminoethyl methacrylate hydrochloride was used as the monomer and 10 g of 1-(acryloyloxy)-3-(methacryloyloxy)-2-propanol was used as the crosslinker in Step 1.

### Comparative Example 2

### Step 1. Preparation of Coating Composition

A coating composition was prepared by dissolving 30 g of acrylamide monomer, 100 g of 1,3-butanediol dimethacrylate and 100 g of 1-(acryloyloxy)-3-(methacryloyloxy)-2-propanol as crosslinkers, and 1 g of 2-hydroxy-2-methylpropiophenone as a photoinitiator in 100 g of toluene (purity ≥99%) with stirring at 1000 rpm for 10 minutes.

### Step 2. Formation of Coating Layer

The coating composition prepared in Step 1 was applied onto a 50 μm thick polyethylene (PE) film (substrate film) by bar coating, followed by UV curing at 1,000 mJ/cm² to form a coating layer having a thickness of 15 μm.

### Step 3. Immobilization of Linker for Organic Synthesis Reaction

The linker for the organic synthesis reaction was immobilized on the coating layer in the same manner as in Step 3 of Example 3.

### Reference Example 2

A substrate for solid-phase organic synthesis was prepared in the same manner as in Example 3, except that, instead of the CuS nanoparticles (average particle diameter: 3 nm and 150 nm) synthesized in Preparation Examples 2-1 and 2-2, respectively, 0.2 g of silver (Ag) nanoparticles (average particle diameter: 60 nm) synthesized by dissolving 10 mL of silver nitrate (AgNO₃) solution (25 mM) and 10 mL of tri-sodium citrate dihydrate solution (1 wt %) in 80 mL of water, followed by reaction for 15 minutes and cooling, was used in Step 1.

### Reference Example 3

A substrate for solid-phase organic synthesis was prepared in the same manner as in Example 3, except that 0.2 g of CuS (average particle diameter: 3 nm) synthesized in Preparation Example 2-1 was used in Step 1.

### Reference Example 4

A substrate for solid-phase organic synthesis was prepared in the same manner as in Example 3, except that 0.2 g of CuS (average particle diameter: 150 nm) synthesized in Preparation Example 2-2 was used in Step 1.

### Preparation Example 3. Preparation of Substrate C for Solid-Phase Organic Synthesis Example 5

An aluminum paste was prepared by dispersing 75 wt % of aluminum (Al) having an average particle diameter (d₅₀) of 3-5 μm in butyl carbitol acetate (BCA) together with a dispersing agent. The prepared aluminum paste was screen-printed once onto the opposite side of a 50 μm thick polyethylene (PE) film (substrate film), on one side of which an Fmoc-Rink linker was chemically bonded, followed by drying and curing at 65° C. for 10 minutes to form an aluminum-based induction heating layer having a thickness of 20±5 μm.

### Example 6

A 30 μm thick aluminum foil was attached as an induction heating layer to the opposite side of a 50 μm thick polyethylene (PE) film (substrate film), on one side of which an Fmoc-Rink linker was chemically bonded.

### Comparative Example 3

A 50 μm thick polyethylene (PE) film, in which an Fmoc-Rink linker was chemically bonded to one side, was used as the substrate for solid-phase organic synthesis. No induction heating layer was formed on the other side of the substrate film.

### Reference Example 5

An aluminum-based induction heating layer was formed on the other side of a 50 μm thick polyethylene (PE) film (substrate film), to which an Fmoc-Rink linker was chemically bonded on one side. An aluminum paste was prepared by dispersing Al powder (average particle diameter (d₅₀): 3˜5 μm, 75 wt %) in a dispersion medium of butyl carbitol acetate (BCA), and the paste was applied three times using screen printing. The printed layer was then dried and cured at 65° C. for 10 minutes to form an induction heating layer having a thickness of 40±5 μm.

### Experimental Example: Simulated Organic Synthesis Reaction

In a conventional solid-phase peptide synthesis reaction, a solid-phase resin having linkers immobilized on its surface is immersed in a solution within a batch reactor, and the reaction is carried out by heating the solution. In such a case, the heated reaction solution cannot be reused and must be discarded after use.

In contrast, the substrate for solid-phase organic synthesis prepared according to the Preparation Example is in the form of a film and, unlike batch reactions, is capable of supporting continuous reactions. Specifically, when the substrate for solid-phase organic synthesis is immersed in a reaction solution within a reactor to perform an organic synthesis reaction, thermal energy required for the reaction can be supplied by induction heating of the substrate, without the need to heat the reaction solution.

Moreover, when using the substrate for solid-phase organic synthesis, it is also possible to supply the thermal energy required for the organic synthesis reaction by spraying the reaction solution directly onto the surface of the substrate and then performing induction heating, without immersing the substrate in the reaction solution.

### Experimental Example 1

The substrates for solid-phase organic synthesis prepared in Preparation Example 1 and Preparation Example 2 were cut into 15 cm×15 cm samples. The surface temperature of each substrate was adjusted to 27.5° C. Thereafter, the heating performance was evaluated by irradiating each substrate with near-infrared (NIR) light for 10 minutes at a distance of 50 cm from the substrate surface.

To induce photothermal conversion of the conductive particles within the induction heating layer, a laser source (MDL-N-808, CNI Laser) emitting at a wavelength of 808 nm and an intensity of 0.75 W/cm² was used. A thermal imaging camera (FLIR E5) was employed to measure the temperature increase.

Table 1 below presents the initial temperature and the maximum temperature reached during 10 minutes of NIR irradiation for each substrate. The results represent the average of 10 measurements.

TABLE 1

| | | | Maximum |
|---|---|---|---|
| | | | temperature |
| | Initial | Maximum | (° C.) − Initial |
| | temperature | temperature | temperature |
| Classification | (° C.) | (° C.) | (° C.) |
| Example 1 | 27.5 | 34.7 | 7.2 |
| Example 2 | 27.5 | 35.4 | 7.9 |
| Example 3 | 27.5 | 45.3 | 17.8 |
| Example 4 | 27.5 | 45.1 | 17.6 |
| Comparative Example 1 | 27.5 | 29.5 | 2 |
| Comparative Example 2 | 27.5 | 28.5 | 1 |
| Reference Example 1 | 27.5 | 32.2 | 4.7 |
| Reference Example 2 | 27.5 | 29.9 | 2.4 |
| Reference Example 3 | 27.5 | 29.2 | 1.7 |
| Reference Example 4 | 27.5 | 30.5 | 3 |

### Experimental Example 2

The substrate for solid-phase organic synthesis prepared in Preparation Example 3 was cut into a 15 cm×15 cm sample. The surface temperature of the substrate was adjusted to 16.8° C.

Thereafter, the substrate was placed on a household induction cooktop (Induction NZ62T3707AK, Samsung Electronics), and the power was turned on. While heating was applied for 40 seconds, the temperature increase was measured using a thermal imaging camera (FLIR E5).

Table 2 below presents the initial temperature and the maximum temperature measured during 40 seconds of induction heating. Each value represents the average of 10 measurements.

TABLE 2

| | | | Maximum |
|---|---|---|---|
| | | | temperature |
| | Initial | Maximum | (° C.) − Initial |
| | temperature | temperature | temperature |
| Classification | (° C.) | (° C.) | (° C.) |
| Example 5 | 16.8 | 57.9 | 41.1 |
| Example 6 | 16.8 | 139.5 | 122.7 |
| Comparative Example 3 | 16.8 | 155.0 | 138.2 |
| Reference Example 5 | 16.8 | 16.9 | 0 |

Referring to Table 2, it was confirmed that in Reference Example 5, in which no induction heating layer was present on the opposite side of the PE film (substrate film), there was little to no temperature change before and after induction heating using the induction cooktop. In contrast, in Comparative Example 3, the ratio of the thickness of the induction heating layer to the thickness of the PE film (substrate film) exceeded 0.5, resulting in excessive heat energy being supplied by the induction heating layer. As a result, partial damage occurred in the substrate for solid-phase organic synthesis.

According to the present invention, the substrate for solid-phase organic synthesis is provided in the form of a film to which at least one linker, at which an organic synthesis reaction is initiated, is fixed. This configuration allows for an increased reactive surface area compared to conventional solid-phase resins used as solid supports for such reactions.

In addition, the length of the organic synthesis reaction achievable via the linker fixed on the substrate film may be relatively longer than that of reactions using conventional solid-phase resins.

Furthermore, the substrate for solid-phase organic synthesis according to the present invention enables a truly continuous and automated organic synthesis process based on a roll-to-roll system, rather than merely linking a series of batch-type reactors as in the prior art.

In conventional batch-type reactions, excess reaction solution is often required to scale up the process. However, as the volume of the reaction solution increases, it becomes increasingly difficult to maintain uniform heating during the organic synthesis reaction. Moreover, the need to dispose of the used reaction solution leads to cost inefficiencies.

In contrast, the continuous and automated organic synthesis process using the substrate of the present invention can achieve higher reaction efficiency than batch-type processes, even when using a relatively small amount of reaction solution.

Additionally, the substrate according to the present invention enables uniform and controlled heat delivery to the reactants via induction heating, eliminating the need to heat the reaction solution in a reactor to supply energy required for the organic synthesis reaction on the substrate.

While the invention has been described with reference to certain embodiments, it will be understood by those of ordinary skill in the art that various modifications, alterations, additions, or deletions may be made without departing from the spirit and scope of the invention as set forth in the claims. Such modifications and variations are to be considered as falling within the scope of the invention.

What is claimed is:

1. A substrate for solid-phase organic synthesis, comprising:
a substrate film; and
at least one linker fixed to one side of the substrate film, wherein an organic synthesis reaction is initiated via the linker,
wherein the substrate film supplies predetermined thermal energy required for the organic synthesis reaction occurring on the substrate film via the linker by induction heating.

2. The substrate for solid-phase organic synthesis of claim 1,
wherein the substrate comprises an induction heating layer that covers at least a portion of one side and the other side of the substrate film and supplies predetermined thermal energy required for the organic synthesis reaction occurring on the substrate film via the linker by induction heating.

3. The substrate for solid-phase organic synthesis of claim 1,
wherein an induction heating layer that supplies predetermined thermal energy required for the organic synthesis reaction occurring on the substrate film via the linker by induction heating is interposed within the substrate film.

4. The substrate for solid-phase organic synthesis of claim 2,
wherein the induction heating layer comprises conductive particles capable of increasing temperature via electromagnetic induction or photothermal conversion.

5. The substrate for solid-phase organic synthesis of claim 4, wherein the conductive particles are coated on at least a portion of one side and the other side of the substrate film.

6. The substrate for solid-phase organic synthesis of claim 4,
wherein the conductive particles comprise at least one selected from metal powders, metal oxides, metal composite oxides, metal sulfides, metal composite sulfides, ceramic powders, and carbonaceous powders.

7. The substrate for solid-phase organic synthesis of claim 5,
wherein the metal comprises at least one selected from aluminum, copper, iron, zinc, nickel, gold, silver, tin, platinum, tungsten, cobalt, lead, silicon, germanium, zinc, titanium, and alloys thereof.

8. The substrate for solid-phase organic synthesis of claim 4,
wherein the induction heating layer comprises first conductive particles and second conductive particles having different average particle sizes.

9. The substrate for solid-phase organic synthesis of claim 8,
wherein the first conductive particles have an average particle size of 100 nm to 100 μm, and the second conductive particles have an average particle size of less than 100 nm.

10. The substrate for solid-phase organic synthesis of claim 2,
wherein the induction heating layer is a metal foil comprising at least one selected from:
(1) a metal (or a metal in powder form) including at least one selected from aluminum, copper, iron, zinc, nickel, gold, silver, tin, platinum, tungsten, and alloys thereof; and
(2) an oxide of at least one selected from aluminum, copper, iron, zinc, nickel, gold, silver, tin, platinum, tungsten, and alloys thereof.

11. The substrate for solid-phase organic synthesis of claim 1,
wherein the substrate comprises:
a first substrate film having at least one linker fixed to one side thereof,
a second substrate film having at least one linker fixed to the other side thereof, and
an induction heating layer interposed between the first substrate film and the second substrate film to supply predetermined thermal energy required for the organic synthesis reaction occurring via the linker fixed on the first and second substrate films by induction heating.

12. The substrate for solid-phase organic synthesis of claim 1,
wherein the organic synthesis reaction is a peptide synthesis reaction or a nucleic acid synthesis reaction.

13. The substrate for solid-phase organic synthesis of claim 1,
wherein the linker is at least one selected from Fmoc-Rink linker, Rink amide linker, PAL linker, PAM linker, BHA linker, Weinreb linker, and Sieber linker.

14. The substrate for solid-phase organic synthesis of claim 1,
wherein the substrate further comprises a non-reactive barrier fixed to one side of the substrate film and provided at a predetermined height adjacent to the linker.

15. The substrate for solid-phase organic synthesis of claim 3,
wherein the induction heating layer comprises conductive particles capable of increasing temperature via electromagnetic induction or photothermal conversion.

16. The substrate for solid-phase organic synthesis of claim 3,
wherein the induction heating layer is a metal foil comprising at least one selected from:
(1) a metal (or a metal in powder form) including at least one selected from aluminum, copper, iron, zinc, nickel, gold, silver, tin, platinum, tungsten, and alloys thereof; and
(2) an oxide of at least one selected from aluminum, copper, iron, zinc, nickel, gold, silver, tin, platinum, tungsten, and alloys thereof.
